(19) United States
(12) Patent Application Publication
(10) Pub. No.: US 2024/0153635 A1
(43) Pub. Date: May 9, 2024

(54) TARGET GENE-BASED DRUG CLINICAL TRIAL SUCCESS RATE PREDICTION MODEL

(71) Applicants: IMMUNOBIOME INC., Pohang-si (KR); POSTECH RESEARCH AND BUSINESS DEVELOPMENT FOUNDATION, Pohang-si (KR)

(72) Inventors: Sang Uk KIM, Pohang-si (KR); Min Hyuk Park, Anyang-si (KR); In Hae KIM, Pohang-si (KR); Chang Wook Park, Seoul (KR)

(21) Appl. No.: 18/447,458

(22) Filed: Aug. 10, 2023

(30) Foreign Application Priority Data
Nov. 8, 2022 (KR) 10-2022-0147881

(51) Int. Cl. G16H 50/20; G16B 15/30; G16B 25/10; G16H 10/20; G16H 70/40

(52) U.S. Cl. CPC G16H 50/20; G16B 15/30; G16B 25/10; G16H 10/20; G16H 70/40

(57) ABSTRACT

According to the present disclosure, it is possible to predict a success or a failure of a clinical trial by reflecting the effect of a drug on a cell population and a human gene. Therefore, it is possible to solve the problems caused by excessive clinical trials, such as excessive use of the drug.

### STATEMENT DESIGNATING GRACE PERIOD INVENTOR DISCLOSURE

The disclosure of the subject matter of this application was made, one year or less before the effective filing date of this claimed invention, by the inventor or joint inventor or by another who obtained the subject matter disclosed directly or indirectly from the inventor or a joint inventor, in Research Square published Aug. 11, 2022, which does not qualify as prior art under 35 USC 102(b)(1)(A) as grace period exception.

### CROSS-REFERENCE TO RELATED APPLICATION

This application claims the benefit under 35 USC 119(a) of Korean Patent Applications No. 10-2022-0147881 filed on Nov. 8, 2022 in the Korean Intellectual Property Office, the entire disclosures of which are incorporated herein by reference for all purposes.

### TECHNICAL FIELD

The present disclosure relates to a target gene-based drug clinical trial success rate prediction model.

### BACKGROUND

A lot of new drugs pass through preclinical trials, but fail in clinical trials due to safety issues. Therefore, the cost of new drug development tends to greatly increase. Also, failure at clinical trials may debase the quality of patients' lives and may cause additional health problems.

Selecting a safe drug target early in the development of a new drug can improve the clinical trial success rate. Conventionally, genetic perturbation (GPA) effects such as CRISPR-Cas9-basd genome editing have been used to assess the safety of a drug target under the assumption that targeting a target gene sensitive to GPA is highly likely to induce serious side effects. This assumption is based on the idea that some genes, which can induce narrow GPA effects are highly likely to be involved in a broad range of biological roles and interactions between major proteins.

However, actually, perturbation effects differ between the cell population and the population. Therefore, clinical trials based on the above assumption may fail due to safety issues of new drug candidates. Even if a specific drug has a small perturbation effect on a specific cell population, the drug may induce a significant perturbation effect on a population, which causes a severe safety problem and results in a clinical test failure.

For example, Sibutramine, an appetite suppressant, did not greatly affect the viability of cell populations. However, the drug has been off the market since 2010 due to serious side effects on humans, such as neurotoxicity, cardiotoxicity, etc. Actually, Sibutramine can suppress a dopamine transporter (SLC6A3), and perturbation on SLC6A3 may not affect the viability of cell populations. However, SLC6A3 has been involved in severe human diseases, such as neuropsychiatric and cardiac disorders.

Meanwhile, Korean Patent No. 10-2274363 discloses a method for establishment of artificial intelligence (machine learning)-based drug development, and U.S. Pat. No. 11,230,741 discloses a method of developing a new patient-specific drug or therapy based on a machine learning algorithm. However, most of prior art references disclose inventions related to processes before and after clinical trials.

Accordingly, there is a demand for a precise prediction technique capable of preventing the occurrence of severe safety problems during development of new drug candidates and clinical trials and thus reducing a huge amount of cost and time from an industrial standpoint and suppressing the occurrence of health risk problems from patients' standpoint.

### PRIOR ART DOCUMENT

### Patent Document

Patent Document 1: Korean Patent No. 10-2274363
Patent Document 2: U.S. patent Ser. No. 11/230,741

### SUMMARY

In view of the foregoing, the present disclosure is conceived to predict a clinical trial success rate in consideration of the discrepancy in perturbation effect of a drug between a preclinical trial on a cell population and a clinical trial on a population. In this regard, the present disclosure is to be construed to include any and all problems that can be understood by a person with ordinary skill in the art.

A first aspect of the present disclosure provides a device for predicting a clinical trial success rate of a new drug by using machine learning, and the device includes an information acquisition unit configured to acquire information about a target gene for a new drug candidate, and a prediction unit configured to predict a clinical trial success rate of the new drug candidate by inputting the information about the target gene into a pre-trained clinical trial success rate prediction model.

A second aspect of the present disclosure provides a method for predicting a clinical trial success rate of a new drug by using machine learning, and the method includes acquiring information about a target gene for a new drug candidate, and predicting a clinical trial success rate of the new drug candidate by inputting the information about the target gene into a pre-trained clinical trial success rate prediction model.

The above aspects are examples only and are to be construed to include any and all aspects that can be understood by a person with ordinary skill in the art.

According to the present disclosure, it is possible to predict a success or a failure of a clinical trial by reflecting the effect of a drug on a cell population and a human gene. Therefore, it is possible to solve the problems caused by excessive clinical trials, such as excessive use of the drug.

The above effects are examples only and are to be construed to include any and all effects that can be understood by a person with ordinary skill in the art.

### BRIEF DESCRIPTION OF THE DRAWINGS

In the detailed description that follows, embodiments are described as illustrations only since various changes and modifications will become apparent to a person with ordinary skill in the art from the following detailed description. The use of the same reference numbers in different figures indicates similar or identical items.

FIG. 1A shows information about 1,064 unapproved drugs and 1,400 approved drugs with respect to 7,695 targets, as sorted according to ATC codes.

FIG. 1B shows histograms analyzing the relationship between a drug and a drug target.

FIG. 1C shows histograms analyzing the relationship between a drug and a drug target.

FIG. 2A is a diagram provided to explain the hypothesis of the present disclosure.

FIG. 2B shows CGE and OGE mapped for approved or unapproved drugs according to an example of the present disclosure. Significance was measured with a two-tailed test.

FIG. 2C shows an overall prediction process according to an example of the present disclosure.

FIG. 2D shows the AUPRC for the result of prediction of clinical trial success rates of unapproved drugs (reaching up to Phases 1, 2 and 3, respectively, in clinical trials) and approved drugs by using OGE+CGE, OGE and CGE according to an example of the present disclosure. Statistical significance of the difference between the AUPRCs was measured with a Mann-Whitney U test.

FIG. 2E shows the locations and number of target genes in the correlation between CGE and OGE according to an example of the present disclosure. In the graph, the y-axis is in loge scale.

FIG. 3 shows the results of predicting clinical trial success rates of specific new drug candidates according to an example of the present disclosure. The results were sorted by clinical trial phase of each drug candidate. Statistical significance between the clinical trial success rates was measured with a Mann-Whitney U test.

FIG. 4A shows the recall and precision in prediction of clinical trial success rates of specific new drug candidates according to an example of the present disclosure (Monte Carlo test, 1,000 times). Red dots connected by a red line represent prediction performance (recall and precision). Statistical significance of each recall and precision was measured with a paired sample t-test.

FIG. 4B shows the recall and precision in prediction of clinical trial success rates of specific new drug candidates according to an example of the present disclosure (Monte Carlo test, 1,000 times). Red dots connected by a red line represent prediction performance (recall and precision). Statistical significance of each recall and precision was measured with a paired sample t-test.

FIG. 5A shows the AUPRC when OGE+CGE, OGE and CGE are used for prediction as prediction performance for unapproved drugs (Phase 1 in clinical trials) and approved drugs (Monte Carlo test, 1,000 times). Statistical significance between the AUPRCs was measured with a Mann-Whitney U test.

FIG. 5B shows the recall and precision for unapproved drugs (Phase 1 in clinical trials) and approved drugs (Monte Carlo test, 1,000 times) according to an example of the present disclosure. Red dots connected by a red line represent prediction performance (recall and precision). Statistical significance of each recall and precision was measured with a paired sample t-test.

FIG. 5C shows the recall and precision for unapproved drugs (Phase 1 in clinical trials) and approved drugs (Monte Carlo test, 1,000 times) according to an example of the present disclosure. Red dots connected by a red line represent prediction performance (recall and precision). Statistical significance of each recall and precision was measured with a paired sample t-test.

FIG. 6A shows a process of classifying risky and safe targets according to an example of the present disclosure. A red dot and a blue dot represent targets showing a bias toward unapproved drugs and approved drugs, respectively, and a gray dot represents a target not showing a bias toward either of unapproved drugs and approved drugs.

FIG. 6B compares each target (drug) classified according to an example of the present disclosure with a drug that fails in an actual clinical trial due to toxicity and a drug with a side effect “Death” (MoleculeNet-ClinTox) through a hypergeometric test.

FIG. 6C compares each target (drug) classified according to an example of the present disclosure with a drug that fails in an actual clinical trial due to toxicity and a drug with a side effect “Death” (K. M. Gayvert et al.) through a hypergeometric test.

FIG. 6D compares each target (drug) classified according to an example of the present disclosure with a drug that fails in an actual clinical trial due to toxicity and a drug with a side effect “Death” (ADReCS) through a hypergeometric test.

FIG. 6E compares each target (drug) classified according to an example of the present disclosure with a drug that fails in an actual clinical trial due to toxicity and a drug with a side effect “Death” (DrugCentral) through a hypergeometric test.

FIG. 7A checks whether a safe drug classified by a method of the present disclosure significantly overlaps an actual toxic drug when the number of risky targets and the number of safe targets are increased by attenuating a reference of statistical significance of drug approval bias according to an example of the present disclosure.

FIG. 7B compares each target (drug) classified according to an example of the present disclosure with a drug that fails in an actual clinical trial due to toxicity and a drug with a side effect “Death” (MoleculeNet-ClinTox) through a hypergeometric test in which a reference of statistical significance of drug approval bias is attenuated.

FIG. 7C compares each target (drug) classified according to an example of the present disclosure with a drug that fails in an actual clinical trial due to toxicity and a drug with a side effect “Death” (K. M. Gayvert et al.) through a hypergeometric test in which a reference of statistical significance of drug approval bias is attenuated.

FIG. 7D compares each target (drug) classified according to an example of the present disclosure with a drug that fails in an actual clinical trial due to toxicity and a drug with a side effect “Death” (ADReCS) through a hypergeometric test in which a reference of statistical significance of drug approval bias is attenuated.

FIG. 7E compares each target (drug) classified according to an example of the present disclosure with a drug that fails in an actual clinical trial due to toxicity and a drug with a side effect “Death” (DrugCentral) through a hypergeometric test in which a reference of statistical significance of drug approval bias is attenuated.

FIG. 8A shows the number of associated drugs per drug target and the degree of drug approval bias (Spearman rho=−0.38) according to an example of the present disclosure. A correlation coefficient was measured by Spearman rank correlation, and the x-axis is in log₁₀ scale, and statistical significance of the difference between the x-axis values was measured with a Mann-Whitney U test.

FIG. 8B shows the number of associated drugs per drug target and the degree of drug approval bias (Spearman rho=−0.43) according to an example of the present disclosure. A correlation coefficient was measured by Spearman rank correlation, and the x-axis is in log₁₀ scale, and statistical significance of the difference between the x-axis values was measured with a Mann-Whitney U test.

FIG. 9A shows genes sorted based on the discrepancy in GPA effects between a cell population and humans according to an example of the present disclosure.

FIG. 9B shows the degree of enrichment of each of risky or safe targets after the genes of FIG. 9A are sorted into N2E.

FIG. 9C shows the degree of enrichment of each of risky or safe targets after the genes of FIG. 9A are sorted into N2N.

FIG. 9D shows the degree of enrichment of each of risky or safe targets after the genes of FIG. 9A are sorted into E2E.

FIG. 9E shows the degree of enrichment of each of risky or safe targets after the genes of FIG. 9A are sorted into E2N.

FIG. 10A shows CGE distribution of risky and safe targets according to an example of the present disclosure. Statistical significance of the discrepancy was measured with a Mann-Whitney U test.

FIG. 10B shows OGE distribution of risky and safe targets according to an example of the present disclosure. Statistical significance of the discrepancy was measured with a Mann-Whitney U test.

FIG. 11A shows the degrees of enrichment of risky and safe targets by using Behan, Fiona M. et al. and OGE according to an example of the present disclosure.

FIG. 11B shows the degrees of enrichment of risky and safe targets by using DepMap and OGE according to an example of the present disclosure.

FIG. 11C shows the degrees of enrichment of risky and safe targets by using hPSCs and OGE according to an example of the present disclosure.

FIG. 11D shows the degrees of enrichment of risky and safe targets by using CGE and pLI(ExAC) according to an example of the present disclosure.

FIG. 11E shows the degrees of enrichment of risky and safe targets by using Behan, Fiona M. et al. and pLI(ExAC) according to an example of the present disclosure.

FIG. 11F shows the degrees of enrichment of risky and safe targets by using DepMap and pLI(ExAC) according to an example of the present disclosure.

FIG. 11G shows the degrees of enrichment of risky and safe targets by using hPSCs and pLI(ExAC) according to an example of the present disclosure.

FIG. 12A checks whether results identical or similar to those of FIG. 11A to FIG. 11G are observed even when a threshold of statistical significance of drug approval bias is attenuated according to an example of the present disclosure.

FIG. 12B checks whether results identical or similar to those of FIG. 11A to FIG. 11G are observed even when a threshold of statistical significance of drug approval bias is attenuated according to an example of the present disclosure.

FIG. 12C checks whether results identical or similar to those of FIG. 11A to FIG. 11G are observed even when a threshold of statistical significance of drug approval bias is attenuated according to an example of the present disclosure.

FIG. 12D checks whether results identical or similar to those of FIG. 11A to FIG. 11G are observed even when a threshold of statistical significance of drug approval bias is attenuated according to an example of the present disclosure.

FIG. 13A is a view showing the result of identifying the effect of the present disclosure by checking how much each of withdrawn drugs targets N2E, N2N, E2E, E2N genes based on a comparison between CGE and OGE according to an example of the present disclosure.

FIG. 13B shows target genes for the withdrawn drugs of FIG. 13A, as sorted by side effect (cardiotoxicity, vascular toxicity, psychiatric toxicity, neurotoxicity).

FIG. 14A shows which genetic pathway an N2E gene involves (or how the N2E gene affects a human body) according to an example of the present disclosure.

FIG. 14B shows which genetic pathway an N2N gene involves (or how the N2N gene affects a human body) according to an example of the present disclosure.

FIG. 14C shows which genetic pathway an E2E gene involves (or how the E2E gene affects a human body) according to an example of the present disclosure.

FIG. 14D shows which genetic pathway an E2N gene involves (or how the E2N gene affects a specific body function) according to an example of the present disclosure.

FIG. 15A shows the components of drug targets and chemical information to be used for predicting a clinical trial success rate according to an example of the present disclosure.

FIG. 15B shows the performance of a clinical trial success rate prediction model using some or all of OGE, CGE, network and expression information according to an example of the present disclosure.

FIG. 15C compares the results of predicting clinical trial success rates of drugs by using drug target information (OGE+CGE+Network+Expression), chemical information (Chemical) or all of them according to an example of the present disclosure.

FIG. 15D shows ATC codes of drugs whose clinical trial success rates are predicted by using only drug target information or chemical information according to an example of the present disclosure.

FIG. 16 shows an improvement in effect of the present disclosure caused by orthogonality between drug target information and chemical information to be used for predicting a clinical trial success rate according to an example of the present disclosure. A vector in the graph indicates the direction and degree of drug approval predictability.

FIG. 17 is a device diagram according to an example of the present disclosure.

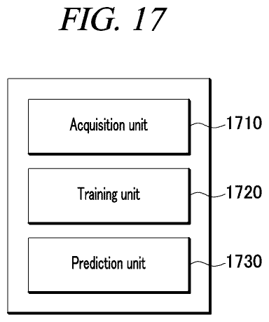

FIG. 18 is a flowchart according to an example of the present disclosure.

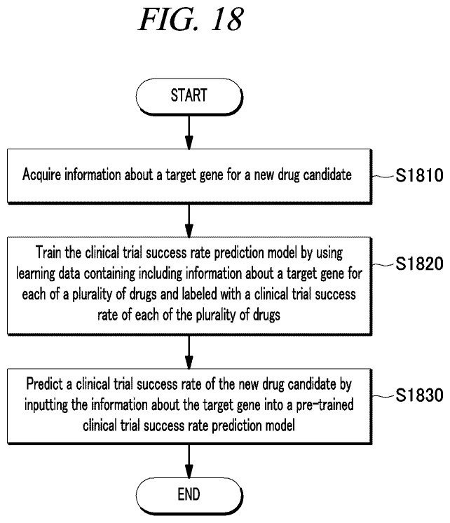

### DETAILED DESCRIPTION

A Hereafter, embodiments of the present disclosure will be described in detail with reference to the accompanying drawings so that the present disclosure may be readily implemented by a person with ordinary skill in the art. However, it is to be noted that the present disclosure is not limited to the embodiments but may be embodied in various other ways. In drawings, parts irrelevant to the description are omitted for the simplicity of explanation, and like reference numerals denote like parts through the whole document.

Throughout the present document, when a member is positioned “on” another member, this includes not only when the member is in contact with the other member, but also when another member is present between the two members.

Throughout the present document, when a part “comprises” a component, this means that other components may be further included rather than excluding the other components unless there is a particular contrary description.

The terms “approxymately” and “substantially” used throughout the document are used in or close to the figure when manufacturing and material tolerances unique to the mentioned meaning are presented, and are used to prevent unscrupulous infringers from unfairly using the disclosure. The term “(doing) step” or “step of˜” to the extent used throughout the present specification does not mean “step for˜”.

Throughout this document, the term “their combination(s)” in the expression of the Makush type refers to one or more mixtures or combinations selected from the group of components described in the Makushi type expression.

Throughout the present specification, the description of “A and/or B” means “A or B, or A and B”.

Throughout this document, the term “subject” includes humans to which drugs are administered to assess the safety in clinical trials after preclinical trials of new drug candidates. The subject is to be construed to include all of animals, such as mouses, rabbits, dogs, cows, horses, sheep, etc., that can be used for drug trials as well as humans.

Throughout this document, the term “clinical test failure” refers to the situation in which a specific drug passes through a preclinical trial, but fails at a clinical trial on a subject group. However, the clinical test failure is not necessarily limited thereto and includes all situations similar to the above-described situation.

Throughout this document, the term “perturbation” or “perturbation effect” refers to a gene-level change caused by a certain substance or stimulation. The perturbation may occur mainly by environmental stimuli, actions of drugs, gene knockdown, etc. For example, the perturbation includes a loss-of-function of a target gene as a result of the action of a drug.

Throughout this document, the term “tolerant” or “loss-of-function tolerant” refers to the situation in which perturbation does not greatly affect the viability of cell populations or populations, such as humans. On the contrary, the term “intolerant” or “loss-of-function intolerant” refers to the situation in which perturbation greatly affects the viability of cell populations or populations, such as humans, because the cell populations or populations cannot accept the effect of perturbation.

Throughout this document, the term “drug-likeness rule” refers to a guideline used to previously select and predict substances similar to a drug from among various compounds and develop a compound suitable for a new drug based on the result of prediction. The components of the drug-likeness rule include a molecular weight, the number of hydrogen bond donors or acceptors, a polar surface area, etc. The drug-likeness rules proposed by Lipinski(Rule of 5; Ro5), Veber and Ghose have been considered as useful guidelines for desirable drugs with low toxicity.

Throughout this document, the term “machine learning” refers to an artificial intelligence application by which a computer program finds a pattern from given data by using an algorithm. In machine learning, a computer is trained to learn from data and improve itself through experience. A machine learning algorithm used in the present disclosure is an example only and is to be construed to include any and all machine learning methods or types which can be used for the present disclosure. For example, the machine learning methods may include supervised learning, unsupervised learning, reinforcement learning, semi-supervised learning, etc. More specifically, the machine learning methods may include all of Naive B ayes Classification, Logistic Regression, Decision tree, Random forest, boosting (XGBoost/ensemble boosting/AdaBoost/Gradient Boost/LightGBM/CatBoost, etc.), Perceptron, Support Vector Machine, Quadratic classifiers, clustering (K-means clustering, Bayesian network clustering, etc.), and the like, but are not limited thereto.

Throughout this document, the term “preclinical trial” refers to a test to check whether a new drug candidate substance has any side effect, toxicity or effect on animals or a cell population before the new drug candidate substance is applied to a population. The preclinical trial includes pharmacokinetics concerned with absorption, distribution and excretion of a drug within the body, and pharmacological research, but is not necessarily limited thereto and is to be construed to include any and all of processes required for trial.

Throughout this document, the term “clinical trial” refers to a test or research conducted on humans to identify the pharmacokinetic, pharmacodynamic, pharmacological and clinical effects of a drug for clinical trial and abnormal responses in order to verify the safety and efficacy of the drug, but is not necessarily limited thereto and is to be construed to include any and all of processes required for trial.

Throughout this document, the term “success rate” refers to a probability of success of a clinical trial, and is calculated as the area under the AUROC of performance prediction results by a model. In the present disclosure, the success rate was set to 0.5, but may be differently set within a range in which accuracy is secured according to needs of a user.

Throughout this document, the term “cellular gene essentiality (CGE)” and the term “organismal gene essentiality (OGE)” refer to the result of calculating the degrees of perturbation effect on a certain gene in a cell population and a population, respectively. This is not essential for a technical concept of the present disclosure. The present disclosure is conceived to predict a clinical trial success rate based on a genetic perturbation effect of a drug on a target.

Throughout this document, the term “protein-protein interaction network (PPIN) information” refers to mathematical representation of a physical contact between proteins in a cell. The contact is very obvious, occurs in a limited binding region between proteins and has a specific biological meaning (specific function).

Hereinafter, embodiments and examples of the present disclosure will be described in detail with reference to the accompanying drawings. However, the present disclosure is not limited to the embodiments, examples and drawings.

A first aspect of the present disclosure provides a device for predicting a clinical trial success rate of a new drug by using machine learning, and the device includes an information acquisition unit configured to acquire information about a target gene for a new drug candidate, and a prediction unit configured to predict a clinical trial success rate of the new drug candidate by inputting the information about the target gene into a pre-trained clinical trial success rate prediction model.

Herein, the information about a target gene may be cellular gene essentiality (CGE) or organismal gene essentiality (OGE).

A second aspect of the present disclosure provides a method for predicting a clinical trial success rate of a new drug by using machine learning, and the method includes acquiring information about a target gene for a new drug candidate, and predicting a clinical trial success rate of the new drug candidate by inputting the information about the target gene into a pre-trained clinical trial success rate prediction model.

All parts common to each aspect apply equally to all the aspects.

### Example 1. Information about Success Rate Prediction Test Using Target Gene Information

(1) Acquisition of Information about Perturbation Effect on Human and Cell Population

The usability of target gene information in predicting a clinical trial success rate was confirmed.

CGE information was derived from fitness scores in the Project SCORE database (Behan, F. M. et al. Prioritization of cancer therapeutic targets using CRISPR-Cas9 screens. Nature 568, 511-516 (2019)). The fitness scores were measured by genome-wide CRISPR-Cas9 screening in 324 cell lines. Genes with an average CGE of 0 or more were identified as essential genes, and genes with an average CGE of less than 0 were identified as non-essential genes.

To identify various metrics of the CGE, the group of fitness genes in Behan, F. M. et al., probability of dependency from the DepMap (20Q4 v2), and the fitness scores of human pluripotent stem cells (hPSCs) were used. Pan-cancer core fitness genes and core fitness genes defined by Behan, F. M. et al. were identified as essential genes, and the others were identified as non-essential genes.

The DepMap screened 789 cell lines by using CRISPR-Cas9 and examined the probability of gene dependency for cell lines by the independent metric compared to the ProjectSCORE. The probability was averaged across 789 cell lines for each gene. Genes with an average probability of 0.5 or more were identified as essential genes, and genes with an average probability of less than 0.5 were identified as non-essential genes.

To conduct a general investigation of cells as well as cancer cell lines, fitness scores for human pluripotent stem cells (hPSCs) in three different media conditions (mouse embryonic fibroblasts feeder cells, laminin and vitronectin) were obtained from Mair, B. et al. The fitness score of the gene for each hPSC was represented as a false discovery rate (FDR). Three fitness scores for each gene were combined through a Fisher's combined probability test with scipy.stats.combine_p values in Python. Genes with a combined FDR of less than 0.01 were identified as essential genes, and genes with a combined FDR of 0.01 or more were identified as non-essential genes.

OGE was derived from LOEUF (loss-of-function observed/expected upper bound fraction) from the gnomAD database (Karczewski, K. J. et al. The mutational constraint spectrum quantified from variation in 141,456 humans. Nature 581, 434-443 (2020)). The LOEUF was estimated by modeling the mutation rate from sequencing data for a human population of up to 140,000 and calculated by comparing the number of expected mutations and the number of observed mutations in the gene. For example, a low LOEUF, which represents a significantly depleted number of observed mutations compared to expected mutations in the gene, is considered to be an intolerant perturbation effect based on negative selection.

The OGE was calculated by subtracting the LOEUF of each gene from 1.996 (the maximum value of LOEUF for all genes) to align the direction with the CGE. According to a threshold of intolerant gene perturbation effects defined by Karczewski, K. J. et al. (LOEUF<0.35), genes with an OGE of 1.646 or more were indicated as essential genes in humans, and genes with an OGE of less than 1.646 were indicated as non-essential genes in humans.

As for the other metrics of OGE, the probability of being loss-of-function intolerance (pLI) from the ExAC database (Lek, M. et al. Analysis of protein-coding genetic variation in 60,706 humans. Nature 536, 285-291 (2016)) was used. Genes with a pLI of 0.9 or more were identified as essential genes, and genes with a pLI of less than 0.9 were identified as non-essential genes.

(2) Mapping of CGE and OGE on Drug

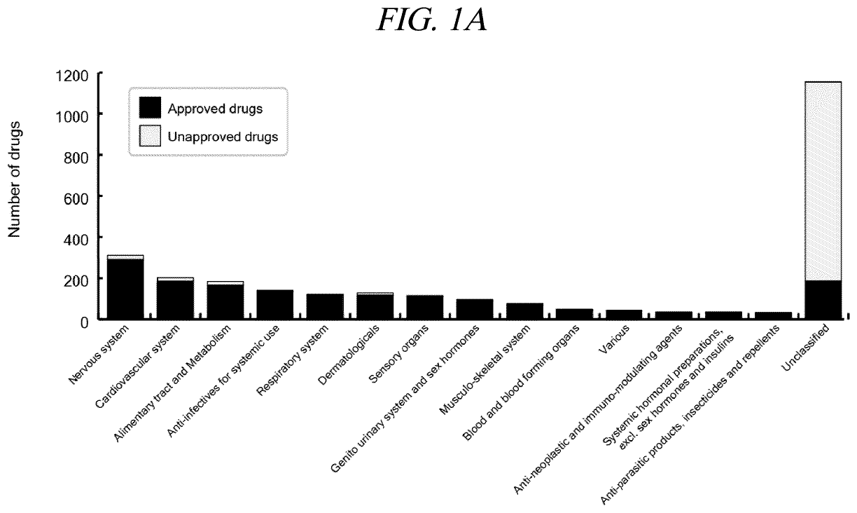

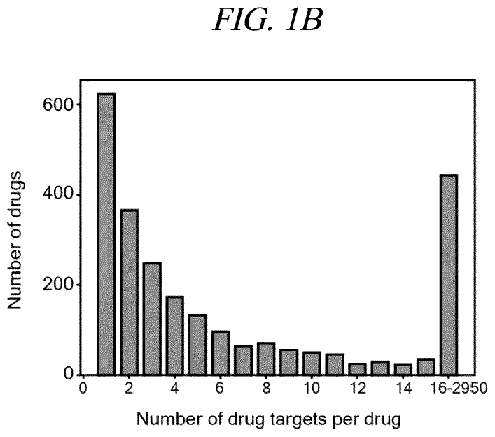

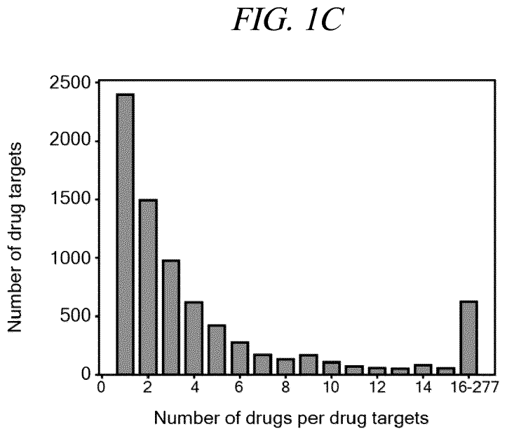

Information about 2,464 drugs for which clinical trials ended at one of phases 1 to 4 was obtained from the ChEMBL database (v30; Mendez, D. et al. ChEMBL: towards direct deposition of bioassay data. Nucleic Acids Res. 47, D930-D940 (2019)). Drugs from phase 4 were identified as approved drugs, and drugs from phases 1, 2 and 3 were identified as unapproved drugs. Drugs used in cancer therapy were excluded because of the inherent cytotoxicity. The molecular type was limited to small molecules. These exclusion criteria were defined by Nguyen et al. (Nguyen, P. A., Born, D. A., Deaton, A. M., Nioi, P. & Ward, L. D. Phenotypes associated with genes encoding drug targets are predictive of clinical trial side effects. Nat. Commun. 10, 1579 (2019)) and Duffy, Aine (Duffy, A. et al. Tissue-specific genetic features inform prediction of drug side effects in clinical trials. Sci. Adv. 6, eabb6242 (2020)). Drug-target interaction information was obtained the STITCH 5 database (Duffy, A. et al. Tissue-specific genetic features inform prediction of drug side effects in clinical trials. Sci. Adv. 6, eabb6242 (2020)) (combined interaction score>=700). Finally, information about 1,064 unapproved drugs and 1,400 approved drugs were compiled with respect to 7,695 targets (see FIG. 1A and FIG. 1B). Drugs were sorted according to Anatomical Therapeutic Chemical (ATC) codes.

The CGE and OGE were mapped for each drug by averaging the CGE and OGE of targets of each drug. All drugs used in an example of the present disclosure were filtered to cover the CGE and OGE of 90% or more of the drug targets.

(3) Machine Learning Procedure

A random forest classifier was used as a clinical trial success rate prediction model and implemented by using Scikit-learn (v0.24.2) in Python. The classifier constructed 1,000 trees with default settings. To train the clinical trial success rate prediction model, the CGE and OGE of drugs against drug status in clinical trials were used. To perform a valid measurement of prediction performance, Monte Carlo cross-validation (1,000 times) was performed to randomly split a dataset into training sets (90%) and test sets (10%). A clinical trial success rate of a new drug candidate was calculated by averaging the calculated probabilities of the clinical trial success rate prediction model for the test sets in 1,000 cross-validations. Drugs with an average approval probability of 0.5 or more may be identified as approved drugs, and drugs with an average approval probability of less than 0.5 may be identified as unapproved drugs. To analyze the prediction performance of the clinical trial success rate prediction model, the area under the precision-recall curve (AUPRC) of the prediction results of the test sets was measured.

### Example 2. Explanation of Success Rate Prediction Test Using Target Gene Information

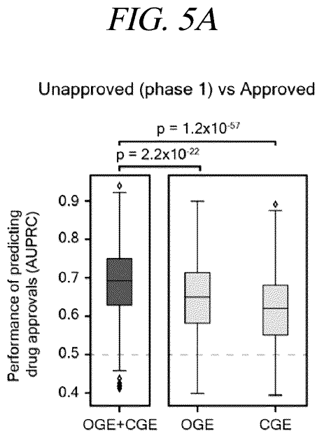

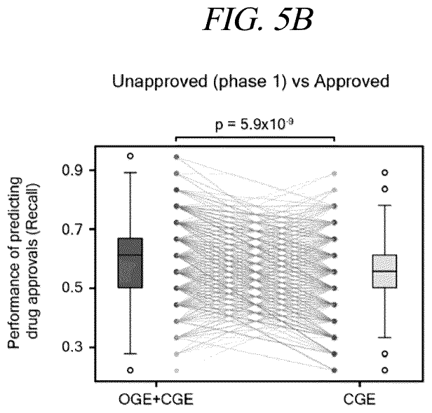

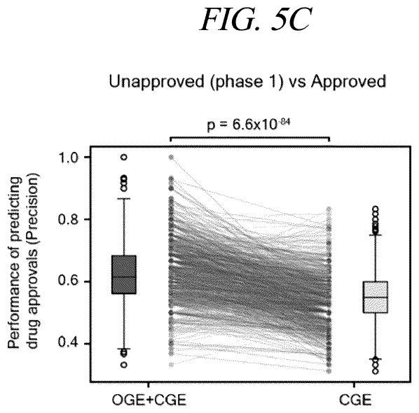

The present inventors conceived that a cause of clinical trial failure of a drug was based on a gene response difference between a preclinical model and a clinical population. The present inventors hypothesized that drugs, which failed in clinical trials, tend to be related to genes with tolerant perturbation effects in cell populations and intolerant perturbation effects in populations such as humans (see FIG. 5A). Herein, the gene perturbation effects on a cell population can be measured by using CRISPR-Cas9 knockouts. However, the gene perturbation effects on a population cannot be measured by using the above-described knockouts. Alternatively, the gene perturbation effects on the population can be estimated by modeling the loss-of-function mutation rates on genes using exome and genome sequencing data from a large population.

Genes with intolerant perturbation effects are conserved in the population for a long time, and, thus, genes with low loss-of-function mutation rates may be considered intolerant to perturbation effects. In contrast, genes with high loss-of-function mutation rates may be considered tolerant to perturbation effects. In the present disclosure, this assumption was used to assess the safety of drugs and therapies in the populations.

(1) Cge/Oge

To identify the association between the discrepancy in drug perturbation effects in the cell population and the population and drug approval in clinical trials, cellular gene essentiality (CGE) and organismal gene essentiality (OGE) were calculated for 17,662 genes in an example of the present disclosure (see Example 1-(1)). Genes with high CGE and OGE scores are essential genes intolerant to perturbation effects in the cell population and the human population, and genes with low CGE and OGE scores are non-essential genes.

(2) Cell Viability Test and Comparison

Drug approval statues were compiled from the ChEMBL database with 7,695 targets for 2,464 drugs (1,064 unapproved drugs and 1,400 approved drugs) from the STITCH 5 database, and the CGE and OGE of drug targets were matched with drugs (see Example 1-(2)). Since the unapproved and approved drugs had passed cell viability tests in preclinical trials, medians of CGE for the unapproved and approved drugs were significantly lower than random distribution (see FIG. 5B; median of CGE for the unapproved drugs=−2.08, Two-tailed test P=5.3×10⁻⁵⁸; P=6.3×10⁻⁸¹). However, a median of OGE for the unapproved drugs was significantly higher than random distribution (median of OGE for the unapproved drugs=1.23, P=5.3×10⁻³⁶), whereas a median of OGE for the approved drugs was significantly lower than random distribution (median of OGE for the approved drugs=1.11, P=3.1×10⁻³³). This result indicates that the unapproved drugs that passed the cell viability tests frequently showed intolerant effects on humans.

Given that drug approvals in clinical trials were associated with the discrepancy in drug target perturbation effects between the cell population and the human population, the present inventors hypothesized that this discrepancy may serve as an important feature for predicting drug approvals in clinical trials.

(3) Establishment of Machine Learning Prediction Model

To verify the hypothesis, the present inventors created a clinical trial success rate prediction model as a machine learning classifier to predict the probabilities of drug approvals using the OGE and CGE of the drug targets (see FIG. 5C). Also, the present inventors created a two stand-alone classifier using one of the OGE or CGE to assess the prediction model of the present disclosure.

The area under the precision-recall curve (AUPRC) of the clinical trial success rate prediction model of the present disclosure using the discrepancy between the CGE and the OGE of the drug targets (OGE+CGE) was 0.70±0.002 (see FIG. 5D; mean value±95% confidence interval), which was considerably higher than the AUPRC of the two stand-alone classifier using one of the OGE or CGE (0.62±0.002, 0.60±0.002, Mann-Whitney U test, P=4.4×10⁻²²¹, P=2.0×10⁻²⁷⁵).

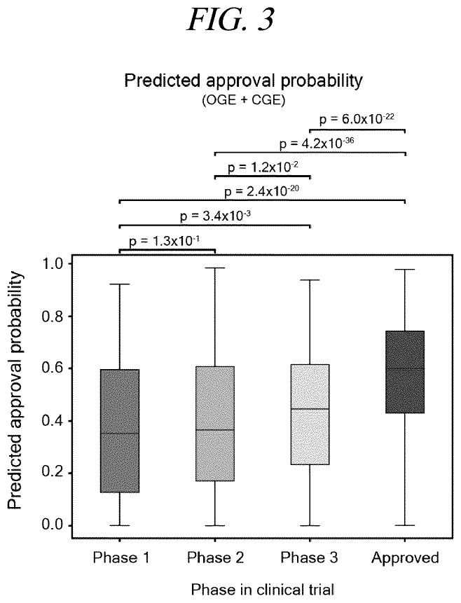

Also, the accuracy of the classifier of the present disclosure was confirmed by assessing the correlation between the approval probability predicted according to the present disclosure and clinical trial phases of the drugs. In general, the probability of approval in the early clinical phases is lower than that in the late clinical phases. Actually, it was observed that drugs with low predicted approval probabilities were associated with the early clinical phases, whereas drugs with high predicted approval probabilities were associated with the late clinical phases (see FIG. 3 and Example 1-(3)).

That is, the clinical trial success rate prediction model shows a remarkably accurate prediction performance and thus can be actually used to predict a clinical trial success rate.

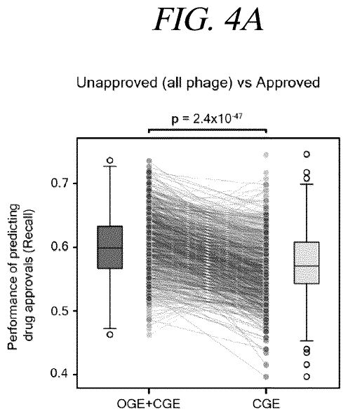

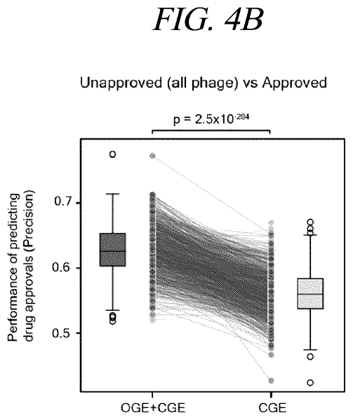

Further, the use of the clinical trial success rate prediction model of the present disclosure can reduce drug attrition rates in clinical trials. Drugs regarded as safe after the cell viability tests have frequently failed in clinical trials due to intolerance to perturbation effects on the humans. The clinical trial success rate prediction model of the present disclosure significantly reduced false predicted approvals of drugs, which were initially predicted as approved drugs based on the gene perturbation effects on the cell population but eventually unapproved. The recall (defined in the present disclosure as the fraction of accurately predicted unapproved drugs among the unapproved drugs) when a clinical trial success rate was predicted using the OGE and CGE was considerably lower than the recall when a clinical trial success rate was predicted using only the CGE (see FIG. 4A; Paired sample t-test; P=2.4×10⁻⁴⁷).

Also, the fraction of approved drugs, which were falsely predicted as unapproved drugs, was also reduced. Precision defined as the fraction of predicted unapproved drugs among the unapproved drugs when a clinical trial success rate was predicted using the OGE and CGE was improved compared to the precision when a clinical trial success rate was predicted using only the CGE (see FIG. 4B; P=2.5×10⁻²⁸⁴).

Predicting drug approvals in clinical phase 1 is necessary to confirm the predictive performance of the clinical trial success rate prediction model (using the OGE and CGE) of the present disclosure for drug safety assessment. This is because drug failure due to safety problems accounts for the largest proportion. A first safety study of healthy volunteers is performed in clinical phase 1. Even when the classifier of the present disclosure was trained with only drugs which were not approved in clinical phase 1 or were finally approved, the prediction accuracy when using the OGE and CGE was improved compared to the prediction accuracy when using the CGE only, and the number of drugs falsely predicted as approved drugs by the CGE was significantly reduced. This suggests that the classifier of the present disclosure performs more precise and excellent prediction on drug safety by reflecting the OGE as intolerant perturbation effects on populations (see FIG. 5A, FIG. 5B and FIG. 5C).

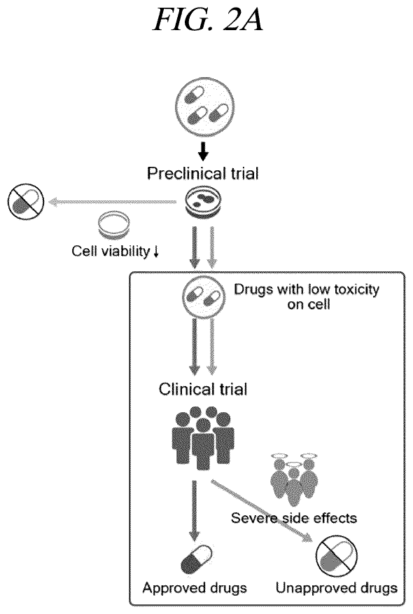

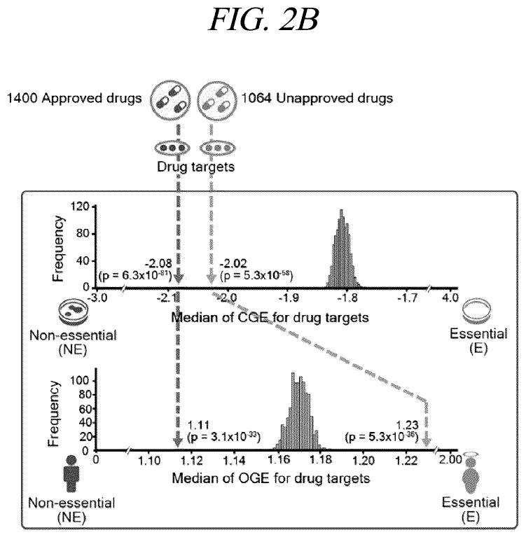

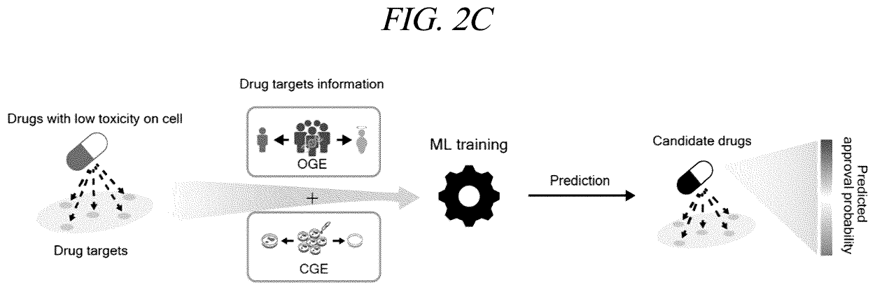

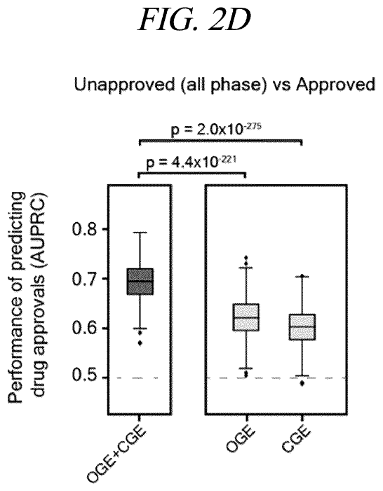

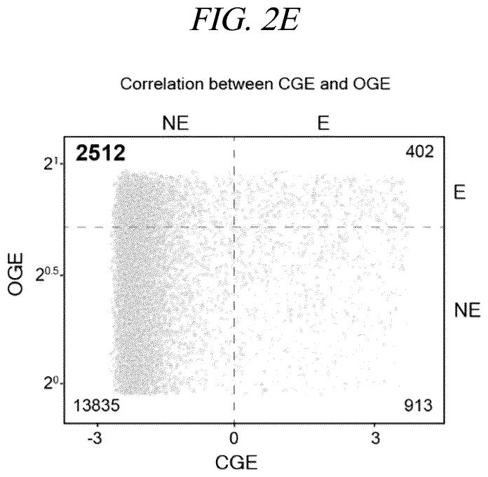

A significant improvement in the predictive performance of the clinical trial success rate prediction model of the present disclosure was possible by using the discrepancy in drug target perturbation effects between a cell population and a population. According to the present disclosure, there were found genes with tolerant perturbation effects on the cell population and intolerant perturbation effects on humans. The use of these genes can contribute to prediction of drug approvals based on the association between the cell population/human discrepancy and drug approvals. The number of the genes was 2,512 and accounts for about 14% of 17,662 genes (see FIG. 2E). Further, the OGE showed a weak correlation with the CGE (Spearman's rho=0.08), which suggests that both the OGE and the CGE used for prediction are key information for predicting drug approvals in clinical trials.

### Example 3. Verification Using Various Metrics

(1) N2E, N2N, E2E, E2N

To identify the association between gene sets (risky/safe targets, N2E, N2N, E2E and E2N) and drugs with safety problems, drugs that failed due to toxicity in clinical trials, drugs having side effects and withdrawn drugs due to safety issues were investigated.

Information about drugs that failed due to toxicity in clinical trials was obtained from two datasets. The ClinTox dataset in MolecularNet (Wu, Z. et al. MoleculeNet: a benchmark for molecular machine learning. Chem. Sci. 9, 513-530 (2018)) contains drugs that failed due to toxicity in clinical trials. The other dataset of toxic drugs was obtained from Gayvert et al. (Gayvert, K. M., Madhukar, N. S. & Elemento, O. A Data-Driven Approach to Predicting Successes and Failures of Clinical Trials. Cell Chem. Biol. 23, 1294-1301 (2016)). Information about failed or approved drugs was obtained from the two datasets.

Information about drugs having side effects was obtained from the ADReCS (v3.1; Cai, M.-C. et al. ADReCS: an ontology database for aiding standardization and hierarchical classification of adverse drug reaction terms. Nucleic Acids Res. 43, D907-D913 (2015)) and Drug Central (v2021; Avram, S. et al. DrugCentral 2021 supports drug discovery and repositioning. Nucleic Acids Res. 49, D1160-D1169 (2021)) databases. In the Drug Central database, side effects of drugs were provided together with a likelihood of a significant drug-side effect relationship and a threshold of the likelihood. The significant drug-side effect relationship was filtered with a likelihood twice the threshold. Sex specificity of side effects was not considered. Drugs with a side effect, death, were defined by the term “Death” from the Medical Dictionary For Regulatory Activities (MedDRA) (MedDRRA term: “Death”, MedDRA ID: 10011906).

Information about withdrawn drugs due to safety issues was obtained from the ChEMBL database (Hunter, F. M. I. et al. Drug Safety Data Curation and Modeling in ChEMBL: Boxed Warnings and Withdrawn Drugs. Chem. Res. Toxicol. 34, 385-395 (2021)) and drug safety data curated by Onakpoya, I. J. et al. (Onakpoya, I. J., Heneghan, C. J. & Aronson, J. K. Post-marketing withdrawal of 462 medicinal products because of adverse drug reactions: a systematic review of the world literature. BMC Med. 14, 10 (2016)). The reasons for failure were classified based on the organs where side effects such as cardiotoxicity and neurotoxicity occurred. The ChEMBL provided failure classes for side effects. The reasons for failure from Onakpoya., I. J. et al. were manually classified.

All drugs with safety problems were mapped to STITCHS identifiers by using mapping files for external database identifiers from STITCHS and PubChemPy (v1.0.4, https://pypi.org/project/PubChemPy).

(2) Risky and Safe Targets

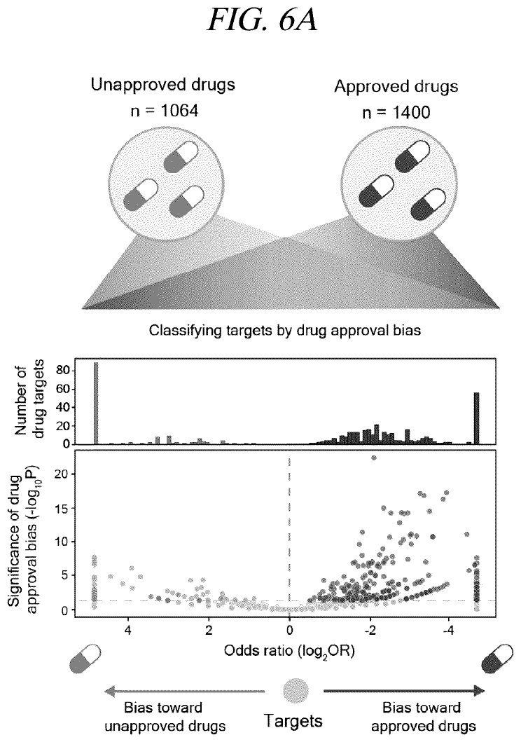

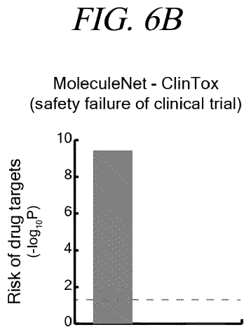

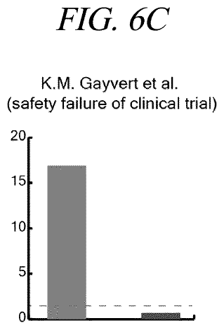

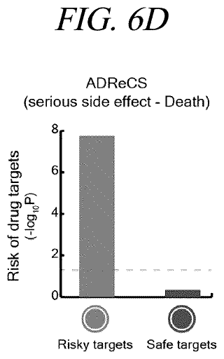

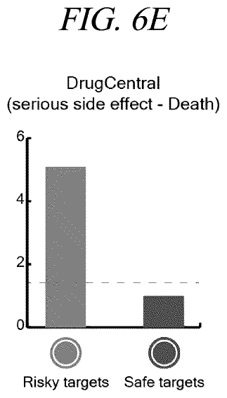

Risky and safe targets were classified to investigate the relationship between the risk of drug targets and the cell population/human discrepancy. The targets were classified by the method according to the present disclosure. Specifically, drug targets associated with unapproved drugs were regarded as risky targets, whereas drug targets associated with approved drugs were regarded as safe targets. Drug approval bias was measured using the odds ratio (OR) and a p value of Fisher's exact test for each drug target. With a p value of less than 0.05, drug targets were classified as risky targets for an OR of more than 1 and safe targets for an OR of less than 1. As a result of measurement, information about 141 risky targets and 313 safe targets was obtained (see FIG. 6A).

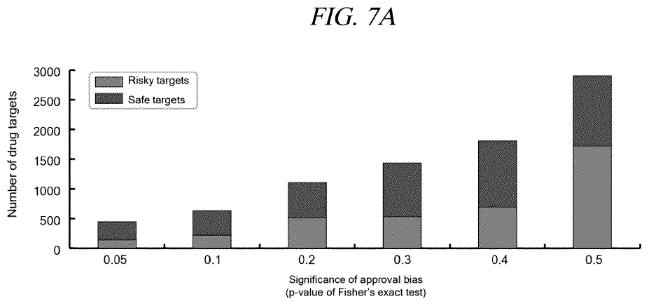

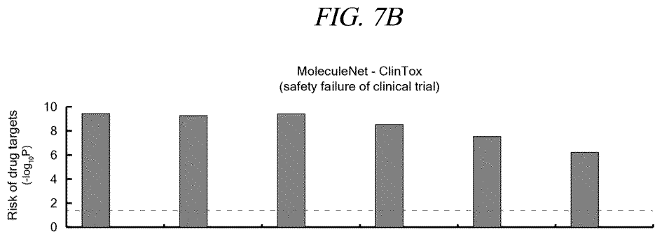

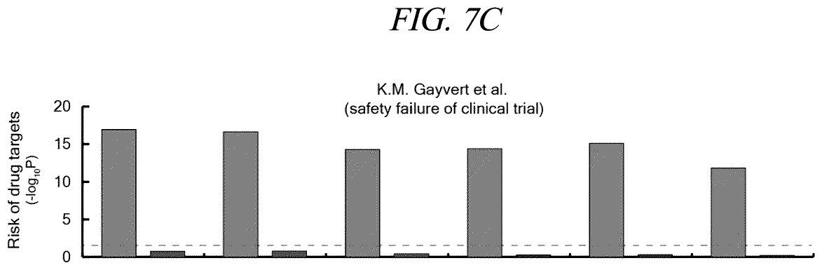

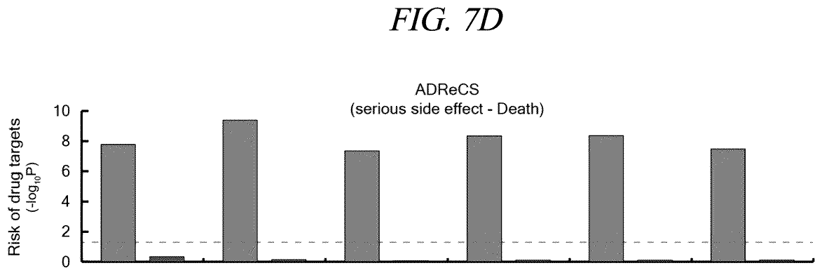

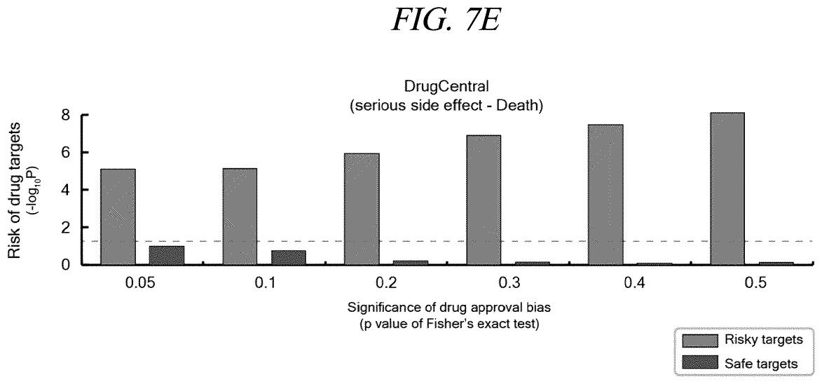

As for each target classified by the method of the present disclosure, the accuracy of the method of the present disclosure was determined by comparing drugs that failed due to toxicity in clinical trials (toxic drugs) with drugs with a side effect “Death”. Risky drugs with risky targets were found to significantly overlap toxic drugs (see FIG. 6B and FIG. 6C; hypergeometric test; P=3.6×10⁻¹⁰; P=1.2×10⁻¹⁷), whereas safe drugs did not significantly overlap toxic drugs (P=1.0; P=0.2). The term “Death” refers to a severe side effect that can lead to death or serious injury. The risky drugs classified by the method according to the present disclosure significantly overlapped the toxic drugs (see FIG. 6D and FIG. 6E; P=1.7×10⁻⁸; P=8.1×10⁻⁶), whereas the safe drugs classified by the method according to the present disclosure did not significantly overlap with the toxic drugs (P=0.5; P=0.1). These results were consistent when a reference of statistical significance of drug approval bias was attenuated to increase the number of risky and safe targets (see FIG. 7A to FIG. 7E), which indicates that the method of the present disclosure can predict a clinical trial success rate well by determining whether a drug is safe or not.

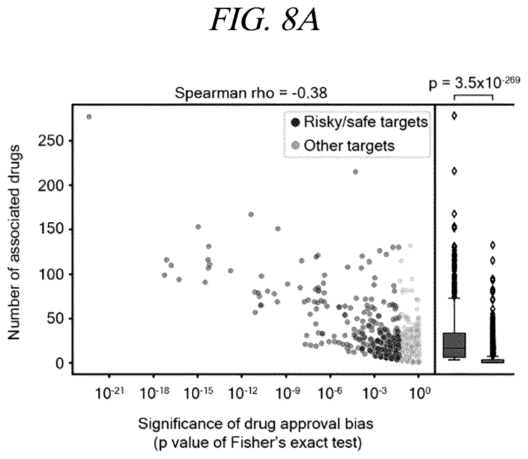

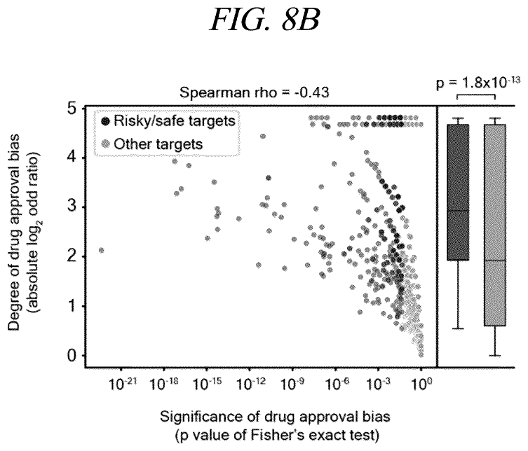

To confirm the effect of the present disclosure, the number of associated drugs per drug target as well as the degree of drug approval bias were examined. A significant bias in drug approvals was associated with a greater number of associated drugs (see FIG. 8A; Spearman rho=−0.38, P=1.6×10⁻²⁶⁹) and a higher degree of drug approval bias (see FIG. 8B; Spearman rho=−0.43, P<1.0×10⁻³⁰⁷). This result indicates that the risky targets or safe targets classified by the method of the present disclosure are explicitly shared by unapproved and approved drugs, respectively. Thus, the risk of drug targets can be assessed by using drug approval bias.

(3) Mapping Between N2E, N2N, E2E and E2N and Risky and Safe Targets

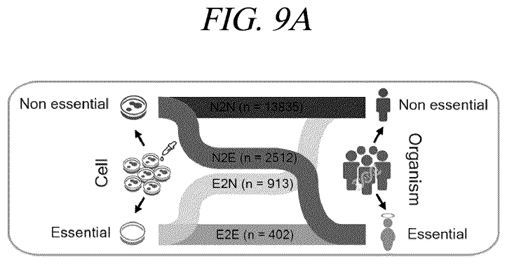

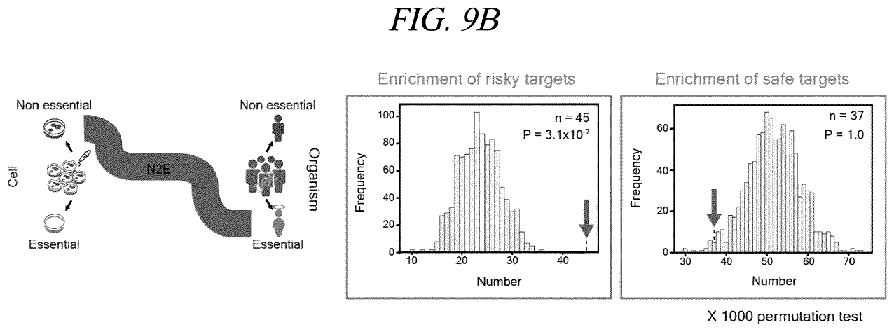

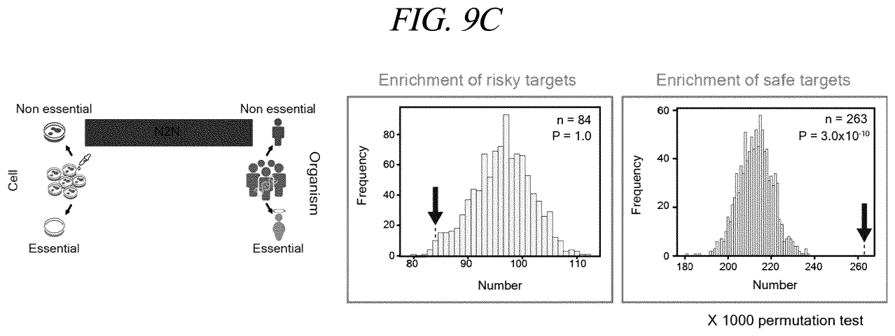

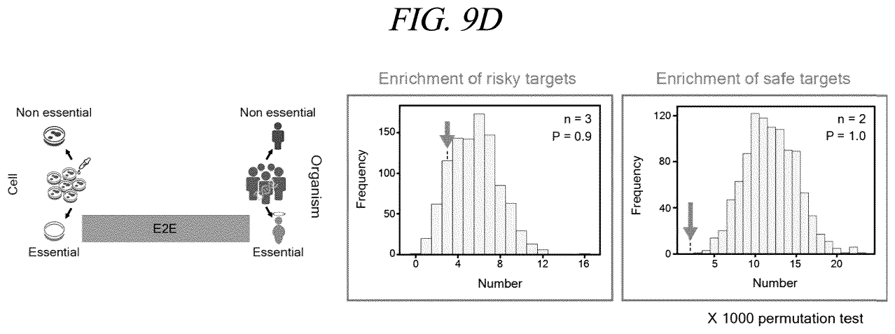

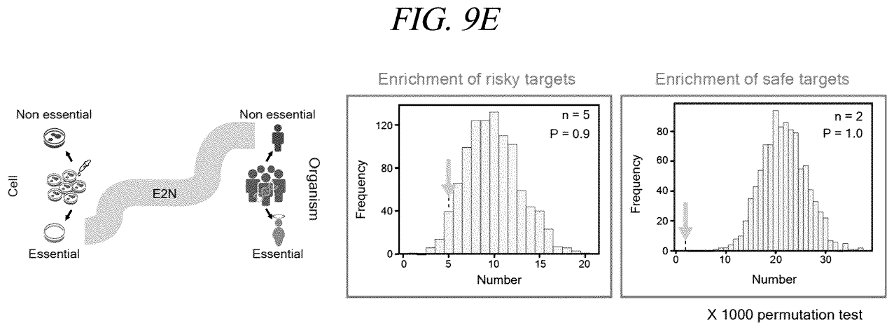

To characterize perturbative genes with risky and safe targets, 17,662 genes were classified into four groups by comparing gene perturbation effects between a cell population and a human population (see FIG. 9A). It was found that 2,512 genes were not essential in cells but essential in humans (N2E), 13,835 genes were not essential in both cells and humans (N2N), 402 genes were essential in both cells and humans (E2E), and 913 genes were essential in cells but not essential in humans (E2N).

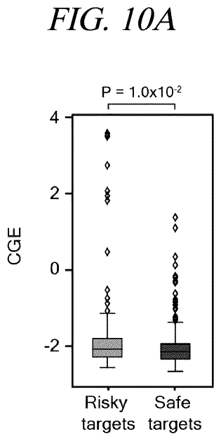

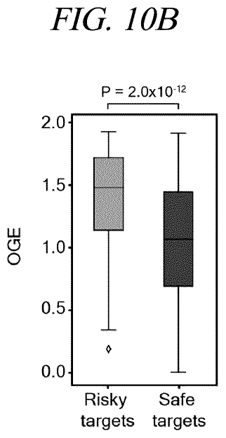

It was found that the risk of drug targets in clinical trials can be characterized by the discrepancy in perturbation effect between the cell population and humans. Particularly, the risky targets were associated with N2E genes, which are tolerant to perturbation effects in the cell population but intolerant to perturbation effects in the human population (see FIG. 9B; One-tailed test; P=3.1×10⁻⁷). OGE of the risky targets was significantly higher than that of the safe targets, and this difference was significantly larger than that in CGE (see FIG. 10A and FIG. 10B; Mann-Whitney U test; P=2.0×10⁻¹²; P=1.0×10⁻¹). Therefore, the risky targets can be characterized by perturbation effects in the population.

In contrast, the safe targets were associated with N2N genes, tolerant to perturbation effects in both the cell population and the humans (see FIG. 9C; P=3.0×10⁻¹⁰). Also, E2E and E2N genes, intolerant to perturbation effects in the cell population, were not associated with the risky or safe targets (see FIG. 9D and FIG. 9E). This suggests that drug targets severely affecting cell viability were excluded from preclinical trials.

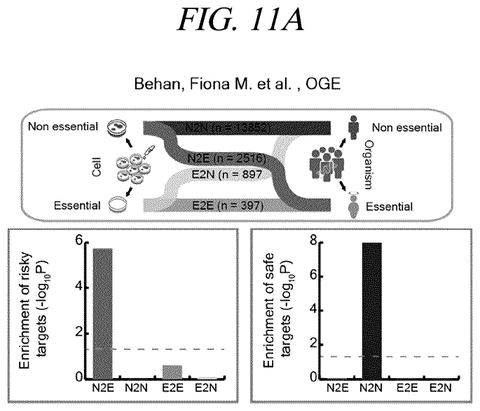

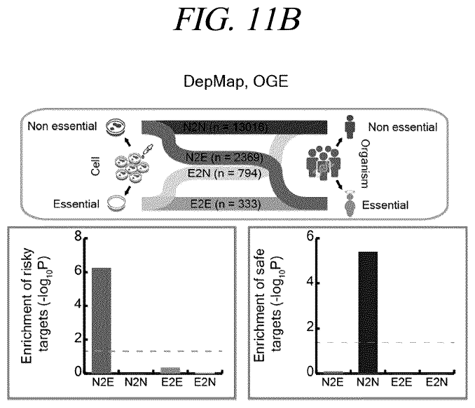

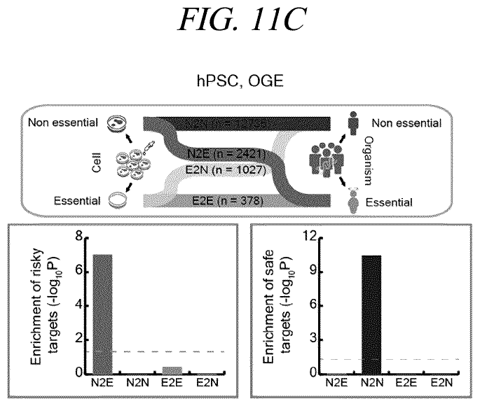

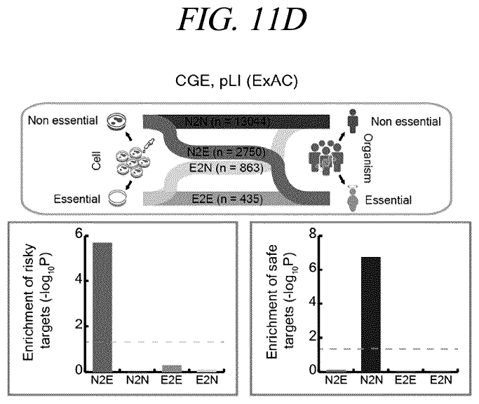

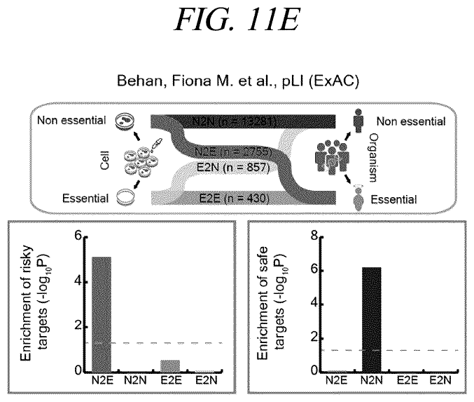

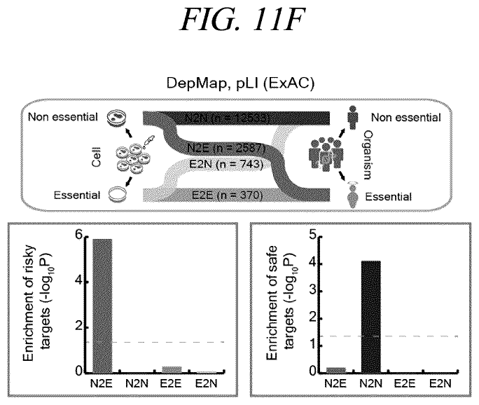

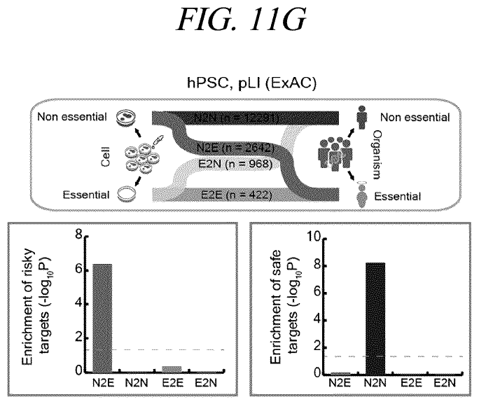

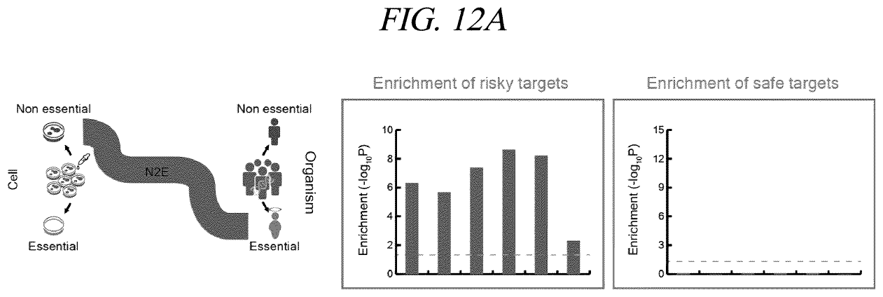

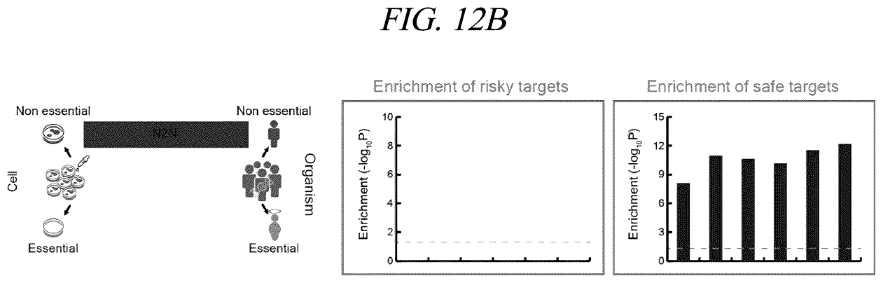

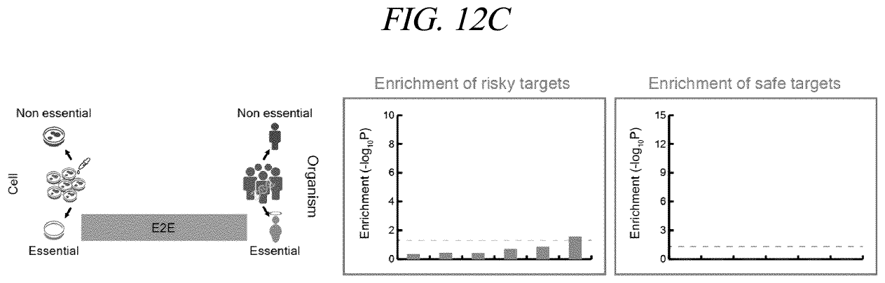

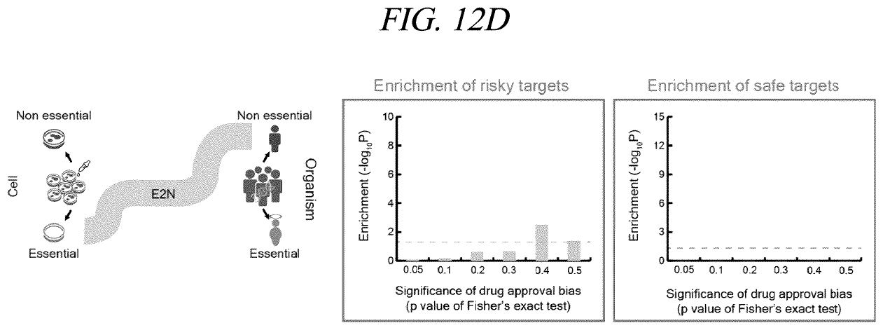

By analyzing various CGE and OGE metrics, it was more clearly confirmed the risky targets were associated with N2E genes, whereas the safe targets were associated with N2N genes. The analysis of OGE was expanded with the probability of being loss-of-function intolerant (pLI) from the ExAC database. As for CGE, the types of fitness genes, the probability of dependency from the DepMap, and fitness cores of human pluripotent stem cells (hPSCs) were investigated from Behan, F. M. et al. Through a similar analysis with other gene essentiality datasets, it was found that the risky and safe targets were significantly enriched in N2E and N2N genes, respectively (see FIG. 11A to FIG. 11G). When a threshold of statistical significance of drug approval bias was attenuated to increase the number of risky and safe targets, significant results were observed consistently (see FIG. 12A to FIG. 12D). This suggests that even when CGE and OGE, which are not biased by increasing the number of risky targets or safe targets, are used for analysis, the same result as shown in the present disclosure is obtained and the effect of the present disclosure is emphasized. Therefore, the risk of drug targets in clinical trials can be assessed by the discrepancy in gene perturbation effects between a cell population and a population.

(4) Comparison Between N2E and Actual Withdrawn Drug

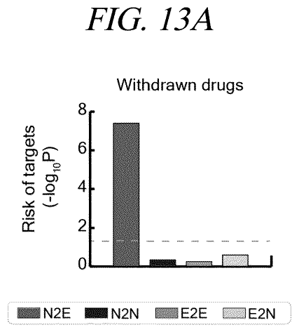

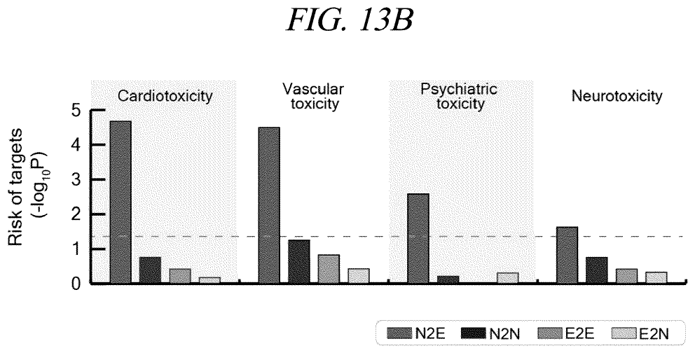

It was confirmed that N2E genes were highly likely to be risky targets with withdrawn drugs having severe side effects. N2E genes were targeted by 139 withdrawn drugs among 200 withdrawn drugs (see FIG. 13A; Hypergeometric test; P=4.0×10⁻⁸). For example, thalidomide used for hyperemesis was withdrawn because of teratogenicity. This is because transcription factor SP1, an N2E gene, is inhibited by thalidomide, and, thus, angiogenesis is disrupted in the embryo. For another example, sibutramine used to treat obesity was withdrawn due to its side effects such as heart attack and stroke. This is because sibutramine inhibits the dopamine transporter (SLC6A3), an N2E gene, and has been implicated in several diseases including neuropsychiatric diseases and heart failure.

It was found that N2E genes were highly likely to induce side effects associated with the central nervous system (CNS) and cardiovascular system, leading to drug withdrawal from markets. These side effects were frequently observed in withdrawn drugs with N2E targets. Withdrawn drugs that induce cardiovascular toxicity, psychiatric and neurotoxicity were significantly associated with N2E targets (see FIG. 13b; P=2.1×10⁻⁵; P=3.2×10⁻⁵; P=2.6×10⁻³; P=2.4×10⁻²). However, drugs with N2N, E2E and E2N targets were not associated with any of the withdrawn drugs having these side effects.

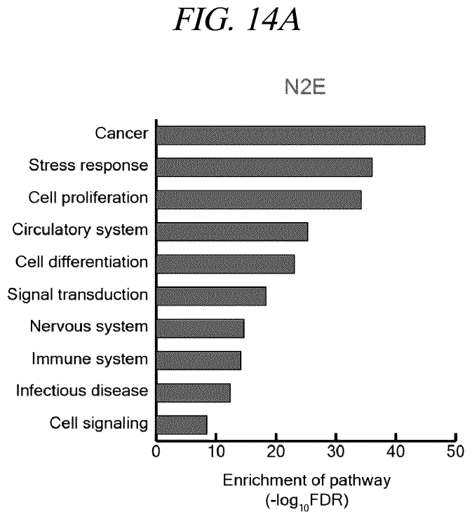

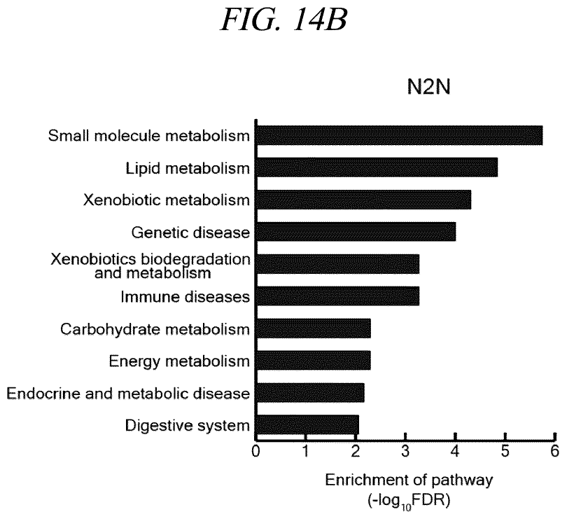

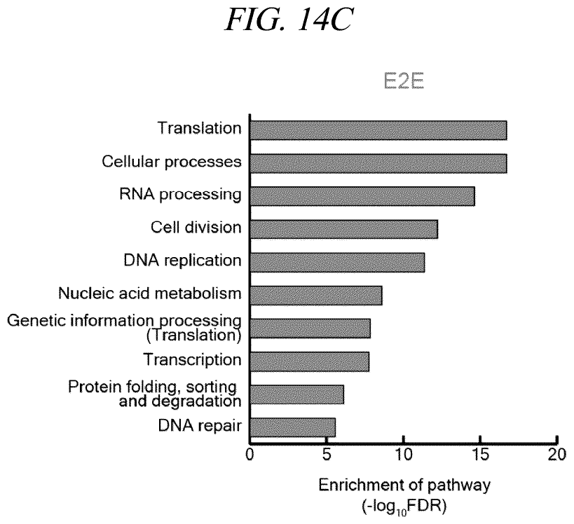

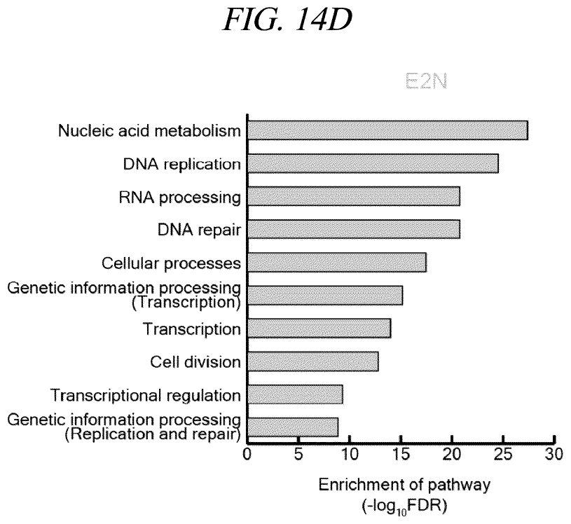

N2E genes are associated with the nervous and circulatory system pathways (see FIG. 14A). For example, N2E genes play an important role in axon guidance. Perturbation effects of axon guidance induce neurological disorders. Angiogenesis is also associated with N2E genes. Cardiotoxicity is induced by perturbation effects of angiogenesis.

Meanwhile, GPCR signaling and metabolism-related pathways are associated with N2N genes (see FIG. 14B). GPCR signaling pathways are targeted by 70% of approved small-molecule drugs, which suggests that N2N genes may be safer drug targets than N2E genes. E2E and E2N genes have been associated with pathways for cellular processes that are essential for cell viability, such as RNA processing, DNA replication and transcription (see FIG. 14C and FIG. 14D).

### Example 4. Success Rate Prediction Test Using Additional Drug Target Information and Chemical Information

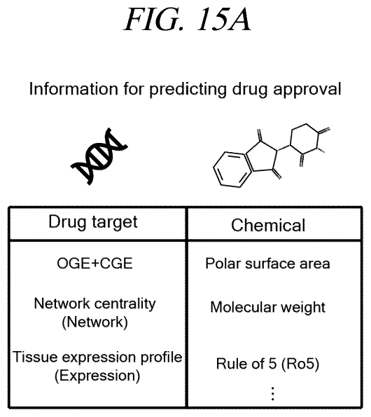

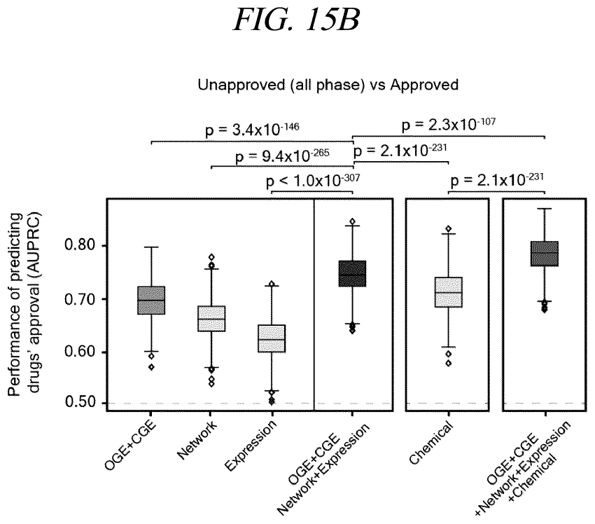

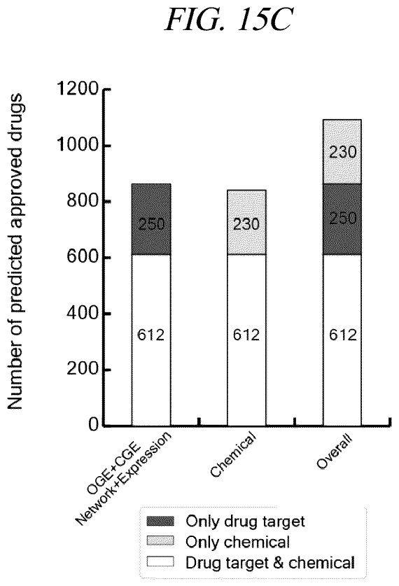

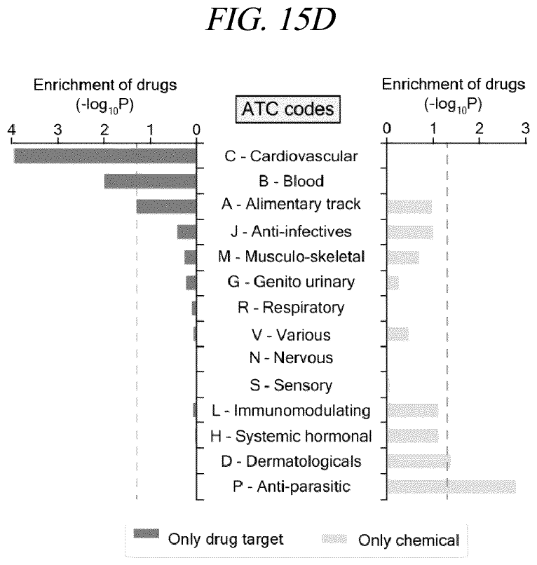

It has been noticed that new drug candidates pass through preclinical trials, but fail in clinical trials due to chemical stability of drugs. The chemical stability of drugs are associated with properties of chemicals constituting the drugs, and includes, for example, a case where a chemical structure of a drug cannot be maintained stably until the drug reaches a target gene. The present inventors confirmed that it is possible to effectively predict a clinical trial success rate when using the above-described information about a target gene together with chemical information. Particularly, when additional drug target information and chemical information are combined with perturbation effects, it is possible to improve the predictive performance of the method of the present disclosure (see FIG. 15A).

For the additional drug target information, protein interaction network (Network) and tissue expression profile (Expression) were used. According to the research, risky drugs tend to target genes having a high degree or betweenness centrality in the protein interaction network and high expression levels or broad expression across tissues, such as the expression of housekeeping genes. For the chemical information, information derived from the drug-likeness rule, such as a molecular weight, the number of hydrogen bond donors or acceptors and a polar surface area, was used. The present inventors combined the above-described information and created models each using some or all of OGE, CGE, network, expression information and chemical information to compare the predictive performance thereof.

The AUPRC of the integrated clinical trial success rate prediction model for predicting drug approvals using OGE, CGE, network, and expression information (OGE+CGE+Expression+Network) was remarkably improved compared to the AUPRC when using OGE and CGE (OGE+CGE; P=3.5×10⁻¹⁴⁶) or using network information (Network; P=9.4×10⁻²⁶⁵) or expression information (Expression; P<1.0×10⁻³⁰⁷) (see FIG. 15B).

Further, the AUPRC of the integrated clinical trial success rate prediction model for predicting drug approvals using chemical information in addition to the above-described information (OGE+CGE+Expression+Network+Chemical) was significantly improved compared to the AUPRC when using OGE, CGE, network and expression information (OGE+CGE+Network+Expression, P=2.3×10⁻¹⁰⁷) or using only chemical information (Chemical, P=2.1×10⁻²³¹).

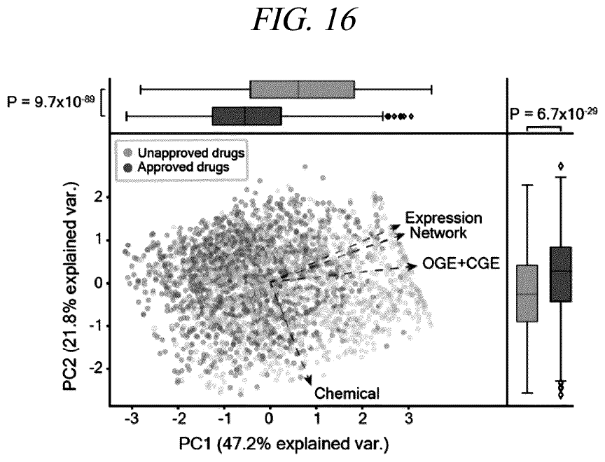

It is considered that prediction of drug approvals using additional information is improved due to the orthogonality between drug targets and chemical information. Chemical information had different explanatory power for prediction from drug target information. The orthogonality between the drug targets and the chemical information was analyzed using principal component analysis (PCA). The vector of chemical information in PCA had a different direction relative to that of drug target information (see FIG. 16). Also, the number of accurately predicted approved drugs was increased by combining drug target and chemical information. The classifiers using one of drug target information (OGE+CGE+Network+Expression) or chemical information (Chemical) accurately predicted 862 and 842 approved drugs, respectively. However, the total number of accurately predicted approved drugs increased to 1,092 when both the drug target information and the chemical information were used (FIG. 15C), which suggests that the accurate prediction of drug approvals can be expanded by combining biological information of drug targets and chemical information of drugs.

Further, the predictive performance may vary depend on the organ or system in which the drug works. Drugs whose clinical trial success rates were predicted using only drug target information or chemical information were investigated according to Anatomical Therapeutic Chemical (ATC) codes. The cardiovascular system (ATC code: C) and blood and blood forming organs (ATC code: B) associated with 250 drugs were predicted by using OGE, CGE, network information and expression information, whereas dermatologicals (ATC code: D) antiparasitic products, insecticides and repellents (ATC code: P) associated with 230 drugs were predicted by using only chemical information (see FIG. 15D). That is, drug target information and chemical information are different from each other in ATC code, and, thus, a broader range of information can be used for prediction. Therefore, it is considered that a clinical trial success rate can be predicted more accurately.

### EXPLANATION OF REFERENCE NUMERALS

1710: Acquisition unit
1720: Training unit
1730: Prediction unit

We claim:

1. A device for predicting a clinical trial success rate of a new drug by using machine learning, comprising:
an acquisition unit configured to acquire information about a target gene for a new drug candidate; and
a prediction unit configured to predict a clinical trial success rate of the new drug candidate by inputting the information about the target gene into a pre-trained clinical trial success rate prediction model.

2. The device of claim 1,
wherein the information about the target gene is a cellular gene essentiality (CGE) and/or an organismal gene essentiality (OGE).

3. The device of claim 1,
wherein the information acquisition unit is configured to further acquire at least one information selected from the group consisting of a protein interaction network, a tissue expression profile and drug-likeness rule component information.

4. The device of claim 3,
wherein the prediction unit is configured to predict the clinical trial success rate of the new drug candidate by further inputting, into the clinical trial success rate prediction model, at least one information selected from the group consisting of the protein interaction network, the tissue expression profile and the drug-likeness rule component information.

5. The device of claim 1, further comprising:
a training unit configured to train the clinical trial success rate prediction model by using learning data including information about a target gene for each of a plurality of drugs and labeled with a clinical trial success rate of each of the plurality of drugs.

6. The device of claim 4,
wherein the training unit is configured to train the clinical trial success rate prediction model through Monte Carlo cross-validation using the learning data.

7. The device of claim 1,
wherein the clinical trial success rate prediction model is trained to improve a clinical trial success rate when the new drug candidate induces tolerant perturbation effects on a cell population and a population.

8. A method for predicting a clinical trial success rate of a new drug by using machine learning, the method comprising:
(a) acquiring information about a target gene for a new drug candidate; and
(b) predicting a clinical trial success rate of the new drug candidate by inputting the information about the target gene into a pre-trained clinical trial success rate prediction model.

9. The method of claim 8,
wherein the information about the target gene is a cellular gene essentiality (CGE) and/or an organismal gene essentiality (OGE).

10. The method of claim 8,
Wherein (a) the acquiring the information about the target gene for the new drug candidate includes further acquiring at least one information selected from the group consisting of a protein interaction network, a tissue expression profile and drug-likeness rule component information.

11. The method of claim 10,
wherein (b) the predicting the clinical trial success rate of the new drug candidate includes predicting the clinical trial success rate of the new drug candidate by further inputting, into the clinical trial success rate prediction model, at least one information selected from the group consisting of the protein interaction network, the tissue expression profile and the drug-likeness rule component information.

12. The method of claim 8, further comprising:
(c) training the clinical trial success rate prediction model by using learning data including information about a target gene for each of a plurality of drugs and labeled with a clinical trial success rate of each of the plurality of drugs.

13. The method of claim 12,
wherein (c) the training the clinical trial success rate prediction model includes training the clinical trial success rate prediction model through Monte Carlo cross-validation using the learning data.

14. The method of claim 8,
wherein the clinical trial success rate prediction model is trained to improve a clinical trial success rate when the new drug candidate induces tolerant perturbation effects on a cell population and a population.
